(19) United States
(12) Patent Application Publication
(10) Pub. No.: US 2023/0280349 A1
(43) Pub. Date: Sep. 7, 2023

(54) Methods of Differentiating a Malignant Tumor from a Non-Malignant Tumor

(71) Applicants: The University of Chicago, Chicago, IL (US); Inova Health System, Falls Church, VA (US); The Henry M. Jackson Foundation for the Advancement of Military Medicine, Inc., Bethesda, MD (US)

(72) Inventors: Ayman Al-Hendy, Hinsdale, IL (US); Hiba Alkelani, Chicago, IL (US); Thomas Patrick Conrads, Reston, VA (US); George Larry Maxwell, Washington, DC (US); Nicholas William Bateman, Leesburg, VA (US); Waleed Barakat, Reston, VA (US)

(73) Assignees: The University of Chicago, Chicago, IL (US); Inova Health System, Falls Church, VA (US); The Henry M. Jackson Foundation for the Advancement of Military Medicine, Inc., Bethesda, MD (US)

(21) Appl. No.: 18/176,909

(22) Filed: Mar. 1, 2023

Related U.S. Application Data
(60) Provisional application No. 63/315,152, filed on Mar. 1, 2022.

(51) Int. Cl. G01N 33/574

(52) U.S. Cl. CPC G01N 33/57488; G01N 2800/7028

(57) ABSTRACT

In aspects, the disclosure provides a method of differentiating a malignant tumor from a non-malignant tumor in a mammal having a tumor comprising determining in a sample from the mammal the level of one or more of: (1) transthyretin (TTR) RNA or protein, (2) dual oxidase 2 (DUOX2) RNA or protein, and (3) G-protein coupled receptor 179 (GPR179) RNA or protein.

### CROSS-REFERENCE TO RELATED APPLICATION

This application claims the benefit of U.S. Provisional Patent Application No. 63/315,152, filed Mar. 1, 2022, which is incorporated by reference herein in its entirety.

### BACKGROUND

Uterine leiomyosarcoma (LMS) is an aggressive tumor associated with high recurrence rates, metastasis, and poor prognosis. It is the most common type of uterine sarcoma. Uterine fibroids (UFs) are benign neoplasms of the myometrium, representing the most common tumors in reproductive aged women globally. Notably, uterine LMS and UFs share several common clinicopathological and imaging features that complicate their differentiation. UF can be treated conservatively, allowing patients to avoid invasive surgery and preserve their fertility, while LMS requires urgent surgery (hysterectomy) and adjuvant chemotherapy.

There is an urgent unmet need to develop early differentiation tools to distinguish these two uterine tumors.

### BRIEF SUMMARY

In aspects, the disclosure provides a method of differentiating a malignant tumor from a non-malignant tumor in a mammal having a tumor, the method comprising: (a) providing a biological sample from the mammal; (b) determining in the sample the level of one or more of: (1) transthyretin (TTR) RNA or protein, (2) dual oxidase 2 (DUOX2) RNA or protein, and (3) G-protein coupled receptor 179 (GPR179) RNA or protein; (c) comparing the level of the one or more RNAs or proteins to the level of the one or more RNAs or proteins in a control biological sample; and (d) determining the tumor is a malignant tumor if (i) the level of TTR RNA or protein in the sample is different than the level of TTR RNA or protein in the control, (ii) the level of DUOX2 RNA or protein in the sample is different than the level of DUOX2 RNA or protein in the control, or (iii) the level GPR179 RNA or protein in the sample is different than the level GPR179 RNA or protein in the control.

In aspects, the disclosure provides a method of differentiating a malignant tumor from a non-malignant tumor in a mammal having a tumor, as described above, wherein (i) the level of TTR RNA or protein in the sample is higher than the level of TTR RNA or protein in the control, (ii) the level of DUOX2 RNA or protein in the sample is higher than the level of DUOX2 RNA or protein in the control, and (iii) the level GPR179 RNA or protein in the sample is different than the level of GPR179 RNA or protein in the control.

In aspects, the disclosure provides a method of treating a mammal having a malignant tumor, the method comprising: (I) determining the tumor is a malignant tumor according to the method described above; and (II) treating the mammal with chemotherapy, radiotherapy, surgery, or as appropriate for the malignant tumor.

Additional aspects of the disclosure are as described herein.

### BRIEF DESCRIPTION OF THE DRAWINGS

FIG. 1 presents micrographs that show the in vivo expression of candidate biomarker transthyretin (TTR) in samples of human uterine leiomyosarcoma (uLMS) and adjacent myometrium (MINI) tissue from 3 different patients (Pt1, Pt2, Pt3), under low and high magnification, using immunohistochemical (IHC) staining. Hematoxylin and eosin (H&E) staining is shown for comparison.

FIG. 2 presents micrographs that show the in vivo expression of candidate biomarker G-protein coupled receptor 179 (GPR179) in samples of human uLMS and adjacent MM tissue from 3 different patients (Pt1, Pt2, Pt3), under low and high magnification, using IHC staining. H&E staining is shown for comparison.

FIG. 3 presents micrographs that show the in vivo expression of candidate biomarker dual oxidase 2 (DUOX2) in samples of human uLMS and adjacent MINI tissue from 3 different patients (Pt1, Pt2, Pt3), under low and high magnification, using IHC staining. H&E staining is shown for comparison.

FIG. 4 shows the expression levels of the three candidate biomarkers present in conditioning media used for growing in vitro cell lines, using enzyme-linked immunosorbent assay (ELISA). The cell lines are: UTSM, immortalized human uterine myometrial smooth muscle; HULM, immortalized human leiomyoma cell line; and SK-UT, uterine leiomyosarcoma. The expression levels of candidate biomarkers transthyretin (TTR; FIG. 4A), dual oxidase 2 (DUOX2; FIG. 4B), and G-protein coupled receptor 179 (GPR179; FIG. 4C) are shown. ns=not significant, *=P<0.05, and ***=P<0.001.

### DETAILED DESCRIPTION

In aspects, the disclosure provides a method of differentiating a malignant tumor from a non-malignant tumor in a mammal having a tumor, the method comprising: (a) providing a biological sample from the mammal; (b) determining in the sample the level of one or more of: (1) transthyretin (TTR) RNA or protein, (2) dual oxidase 2 (DUOX2) RNA or protein, and (3) G-protein coupled receptor 179 (GPR179) RNA or protein; (c) comparing the level of the one or more RNAs or proteins to the level of the one or more RNAs or proteins in a control biological sample; and (d) determining the tumor is a malignant tumor if (i) the level of TTR RNA or protein in the sample is different than the level of TTR RNA or protein in the control, (ii) the level of DUOX2 RNA or protein in the sample is different than the level of DUOX2 RNA or protein in the control, or (iii) the level GPR179 RNA or protein in the sample is different than the level GPR179 RNA or protein in the control.

In aspects, preliminary diagnosis of a malignant tumor can be made from clinical symptomology, by visualization on ultrasound, MM, or similar analyses, or by other known means. In aspects, the malignant tumor is any uterine sarcoma or sarcoma of uterine origin. In aspects, the malignant tumor is uterine leiomyosarcoma (LMS). In aspects, the non-malignant tumor is a uterine tumor. In aspects, the non-malignant uterine tumor is a benign uterine tumor. In aspects, the benign uterine tumor is a uterine fibroid (UF).

In aspects, the biological sample is biological material such as blood serum, uterine tissue, urine, or stool.

In aspects, the control is non-malignant biological material. In aspects, the control is non-malignant biological material of the same type of biological material as the biological sample from the mammal.

In aspects, the differentiation is between a malignant uterine tumor and a benign uterine tumor. In aspects, the malignant uterine tumor is a uterine leiomyosarcoma (LMS) tumor and the benign uterine tumor is a uterine fibroid (UF). In yet other aspects, the malignant uterine tumor is a uterine leiomyosarcoma (LMS) tumor and the benign uterine tumor is a uterine fibroid (UF).

In aspects, the tumor differentiation is pre-operative or post-operative. In aspects, the pre-operative differentiation is for diagnostic purposes (e.g., of uterine leiomyosarcoma and/or uterine fibroid). In aspects, the post-operative differentiation is for prognostic purposes. In aspects, the prognostic differentiation tests for recurrence of LMS.

In aspects, the mammal is a human female.

In aspects, the sample is biological material such as serum, urine, tissue, or stool.

In aspects, the RNA or protein level is of protein (e.g., in serum or urine), and the level is determined by quantitative mass spectrometry. In aspects, the RNA or protein level is of protein (e.g., in serum or urine), and the level is determined by enzyme-linked immunosorbent assay. In aspects, the RNA or protein level is of protein (e.g., in serum or urine), and the level is determined by Western Blot analysis. In aspects, the RNA or protein level is of protein (e.g., in serum or urine), and the level is determined by RIA (radioimmune assay) analysis. In aspects, the RNA or protein level is of protein (e.g., in tissue or stool), and the level is determined by IHC (immunohistochemistry) analysis. In aspects, the IHC determination is performed under various magnification levels. In aspects, the RNA or protein level is of RNA or protein (e.g., in serum or urine), and the level is determined by chromatography. In aspects, the RNA or protein level is of RNA (e.g., in serum or urine), and the level is determined by Northern Blot analysis. In aspects, the RNA or protein level is of RNA (e.g., in serum or urine), and the level is determined by nuclease protection assay (NPA). In aspects, the RNA or protein level is of RNA (e.g., in tissue or stool), and the level is determined by in situ hyhribidization (ISH). In aspects, the RNA or protein level is of RNA (e.g., in serum or urine), and the level is determined by reverse-transcriptase PCR (RT-PCR).

In aspects, the disclosure provides a method of differentiating a malignant tumor from a non-malignant tumor in a mammal having a tumor, as described above, wherein (i) the level of TTR RNA or protein in the sample is higher than the level of TTR RNA or protein in the control, (ii) the level of DUOX2 RNA or protein in the sample is higher than the level of DUOX2 RNA or protein in the control, or (iii) the level GPR179 RNA or protein in the sample is different than the level of GPR179 RNA or protein in the control. In aspects, the level is of RNA. In aspects, the level is of protein.

In aspects, the disclosure provides a method of differentiating a malignant tumor from a non-malignant tumor in a mammal having a tumor, as described above, wherein (i) the level of TTR RNA or protein in the sample is higher than the level of TTR RNA or protein in the control, (ii) the level of DUOX2 RNA or protein in the sample is higher than the level of DUOX2 RNA or protein in the control, and (iii) the level GPR179 RNA or protein in the sample is different than the level of GPR179 RNA or protein in the control. In aspects, the level is of RNA. In aspects, the level is of protein.

In aspects, the disclosure provides a method of treating a mammal having a malignant tumor, the method comprising: (I) determining the tumor is a malignant tumor according to the method as described above; and (II) treating the mammal with chemotherapy, radiotherapy, surgery or as appropriate for the malignant tumor. In aspects, the malignant tumor is a uterine leiomyosarcoma (LMS) tumor. In aspects, the mammal is a human female. Once a determination has been made, management such as choice of chemotherapy, radiotherapy, surgery or other treatment is determined by the nature and the stage of the tumor.

The following includes certain aspects of the disclosure.

1. A method of differentiating a malignant tumor from a non-malignant tumor in a mammal having a tumor, the method comprising:
(a) providing a biological sample from the mammal;
(b) determining in the sample the level of one or more of: (1) transthyretin (TTR) RNA or protein, (2) dual oxidase 2 (DUOX2) RNA or protein, and (3) G-protein coupled receptor 179 (GPR179) RNA or protein;
(c) comparing the level of the one or more RNAs or proteins to the level of the one or more RNAs or proteins in a control biological sample; and
(d) determining the tumor is a malignant tumor if (i) the level of TTR RNA or protein in the sample is different than the level of TTR RNA or protein in the control, (ii) the level of DUOX2 RNA or protein in the sample is different than the level of DUOX2 RNA or protein in the control, or (iii) the level GPR179 RNA or protein in the sample is different than the level GPR179 RNA or protein in the control.

2. The method of aspect 1, wherein the non-malignant tumor is a benign tumor.

3. The method of aspect 1 or 2, wherein the biological sample is serum.

4. The method of any one of aspects 1-3, wherein the differentiation is between a malignant uterine tumor and a benign uterine tumor, wherein the malignant uterine tumor is a uterine leiomyosarcoma (LMS) and the benign uterine tumor is a uterine fibroid (UF).

5. The method of aspect 4, wherein the tumor is determined to be an LMS tumor.

6. The method of any one of aspects 1-5, wherein (i) the level of TTR RNA or protein in the sample is higher than the level of TTR RNA or protein in the control, (ii) the level of DUOX2 RNA or protein in the sample is higher than the level of DUOX2 RNA or protein in the control, or (iii) the level GPR179 RNA or protein in the sample is different than the level of GPR179 RNA or protein in the control.

7. The method of any one of aspects 1-6, wherein (i) the level of TTR RNA or protein in the sample is higher than the level of TTR RNA or protein in the control, (ii) the level of DUOX2 RNA or protein in the sample is higher than the level of DUOX2 RNA or protein in the control, and (iii) the level GPR179 RNA or protein in the sample is different than the level of GPR179 RNA or protein in the control.

8. The method of any one of aspects 1-7, wherein the level is of RNA.

9. The method of any one of aspects 1-7, wherein the level is of protein.

10. The method of any one of aspects 1-9, wherein the differentiation is pre-operative.

11. The method of aspect 9, wherein the level of protein is determined by quantitative mass spectrometry.

12. The method of aspect 9, wherein the level of protein is determined by enzyme-linked immunosorbent assay.

13. The method of any one of aspects 1-12, wherein the mammal is a human female.

14. A method of treating a mammal having a malignant tumor, the method comprising:
(I) determining the tumor is a malignant tumor according to the method of any one of aspects 1-13; and
(II) treating the mammal with chemotherapy, radiotherapy, surgery or as appropriate for the malignant tumor.

### Example 1

### Cell Lines

The immortalized human fibroid cell line (HuLM) is as described in Carney S A et al. (2002) Immortalization of human uterine leiomyoma and myometrial cell lines after induction of telomerase activity: molecular and phenotypic characteristics. Lab Invest. 82(6): 719-728. The cell line is cultured and maintained in phenol red-free, 10% fetal bovine serum Dulbecco's modified Eagle's medium/nutrient mixture F-12 (DMEM/F-12) from Thermo Fisher Scientific (Waltham, Mass., USA) supplemented with L-glutamine, 10% FBS, and 1% antibiotic-antimycotic in 5% CO₂, at 37° C.

The uterine leiomyosarcoma (uLMS) cell line (SK-UT1, ATCC® HTB-114™ from ATCC, Manassas, Va., USA) is cultured and maintained in ATCC-formulated Eagle's Minimum Essential Medium with 10% of fetal bovine serum (Yang Q et al. (2022) The Functional Role and Regulatory Mechanism of Bromodomain-Containing Protein 9 in Human Uterine Leiomyosarcoma. Cells, 11(14): 2160).

### Mouse-Human Proteomics

Quantitative proteomic analysis identifies new candidate biomarkers (Tarney C M et al. (2019) Biomarker panel for early detection of endometrial cancer in the Prostate, Lung, Colorectal, and Ovarian cancer screening trial. American Journal of Obstetrics and Gynecology, 221(5): 472.e1-472.e10).

Briefly, nude mice are inoculated with 2×10⁷ of SK-UT1 human LMS cells (n=5), HuLM UF cells (n=5) or control with no cells (n=5). After 4 weeks, animal blood is collected. Serum is prepared from the collected whole blood and immunodepleted of abundant blood proteins, such as albumin, IgG and transferrin, by chromatography with Multiple Affinity Removal Column Human 14 (Agilent, Santa Clara, Calif., USA). Protein eluates are digested with trypsin using pressure-cycle technology. Peptide digests are labeled with tandem-mass tag (TMT-18) isobaric labels, are fractionated offline by basic reversed-phase liquid chromatography, and are analyzed by high-resolution mass spectrometry on an Orbitrap mass spectrometer (Thermo Fisher Scientific, Waltham, Mass., USA). Conditions of interest are compared, and significantly altered proteins are visualized in heatmaps and by PCA using default settings in the ClustVis web tool (biit. cs. ut. ee/clustvis). Functional prediction analysis is performed using the method provided by Metascape.org. (metascape. org/gp/index. html #/main/step1).

Raw data is searched against a concatenated mouse/human proteome database, and human-specific proteins are identified by ≥2 human-specific peptides. Differential analysis is performed using LIMMA, and significantly (p<0.05) altered candidates exhibiting a minimum±1.5 fold-change are prioritized for downstream analysis.

Quantitative proteomics is used to identify novel serum biomarkers specific for LMS by identifying proteins in serum that vary in their expression levels (amount of RNA and protein), between sera from LMS, UF, and control samples.

Global proteomic analysis quantifies 194 human-specific proteins across samples. Differential analysis identifies six significantly altered proteins that are shared in common between LMS and UFs. The differential analysis also identifies twelve proteins whose expression is unique to cells from benign tumors, and 10 that are unique to cells from malignant tumors. Notably, three proteins are altered only in the LMS samples and include signal peptide sequences. Signal peptides target proteins for secretion from the cell, or target them to the cellular membrane from which they may be shed to the extracellular space. Secreted proteins can be analyzed readily in serum samples prepared from blood collected from animals.

The secreted proteins unique to LMS cells are identified as transthyretin (TTR), dual oxidase 2 (DUOX2), and G-protein coupled receptor 179 (GPR179). The TTR and DUOX2 proteins are upregulated, and GPR179 is downregulated, in LMS serum samples relative to controls. In the LMS serum samples, the TTR and DUOX2 proteins are present at greater levels than the levels of TTR protein and DUOX2 protein in the control sera, while GPR179 protein is present at a lower level than the level of GPR179 protein in the control sera.

### Example 2

### Immunohistochemical (IHC) Analysis

Histological sections from UF tissue and LMS tissue samples are prepared by formalin-fixing and embedding in paraffin, then are used for immunostaining as follows: For Duox2, the antibody is ab97266 Anti-DUOX2 antibody (rabbit polyclonal antibody, Abcam, Waltham, Mass., USA). The slides are stained on Leica Bond RX Automatic Stainer. After Dewax, rehydration and antigen retrieval treatment (epitope retrieval solution II, AR9640, Leica Biosystems) for 20 minutes, anti-DUOXa antibody (1:200) is applied on tissue sections for 60 minutes incubation and the antigen-antibody binding is detected with Bond Polymer Refine Detection without Post Primary (Leica Biosystems, DS9800). The tissue sections are counterstained with hematoxylin and covered with cover glasses.

For TTR, the antibody is Transthyretin Rabbit anti-Human, monoclonal antibody (JM11-43, Invitrogen™, Thermo Fischer Scientific, Waltham, Mass., USA). The slides are stained on Leica Bond RX Automatic Stainer. After Dewax, rehydration and antigen retrieval treatment (epitope retrieval solution II, AR9640, Leica Biosystems) for 20 minutes, anti-Transthyretin Recombinant antibody (1:2000) is applied on tissue sections for 60 minutes incubation, and the antigen-antibody binding is detected with Bond Polymer Refine Detection without Post Primary (Leica Biosystems, DS9800). The tissue sections are counterstained with hematoxylin and covered with cover glasses.

For GPR 179, the antibody is Anti-GPR179 (rabbit, Prestige Antibodies® HPA017885, Sigma-Aldrich, St. Louis, Mo., USA). The slides are stained on Leica Bond RX Automatic Stainer. After Dewax, rehydration and antigen retrieval treatment (epitope retrieval solution II, AR9640, Leica Biosystems) for 20 minutes, anti-GPR179 antibody (1:200) is applied on tissue sections for 60 minutes incubation and the antigen-antibody binding is detected with Bond Polymer Refine Detection without Post Primary (Leica Biosystems, DS9800). The tissue sections are counterstained with hematoxylin and covered with cover glasses. LMS and UF tissues in formalin-fixed paraffin-embedded (FFPE) human uterine tissue samples from three patients are compared using immunohistochemical (IHC) staining specific for each biomarker, under low and high magnification.

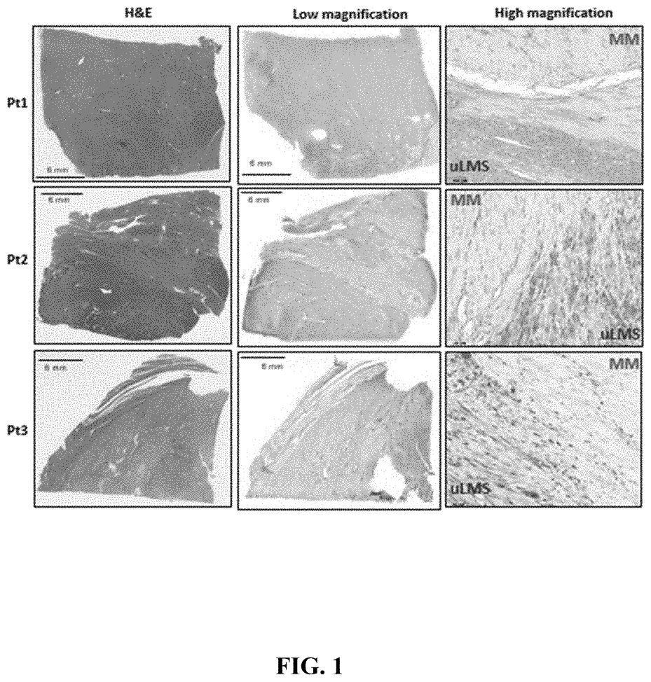

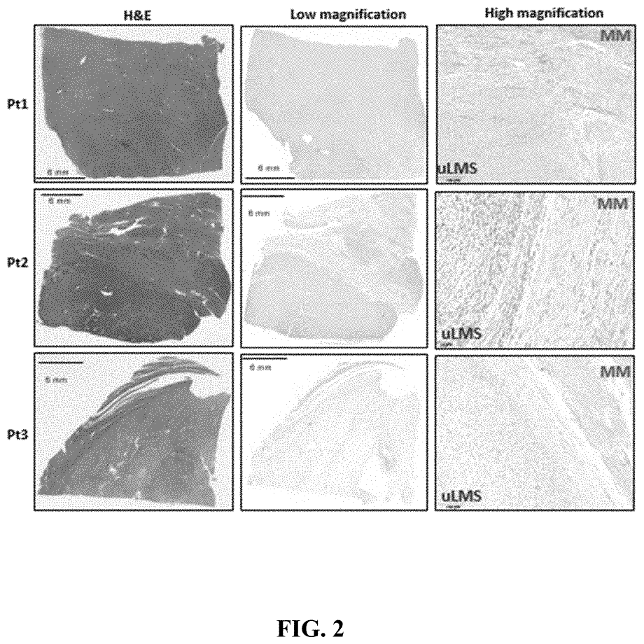

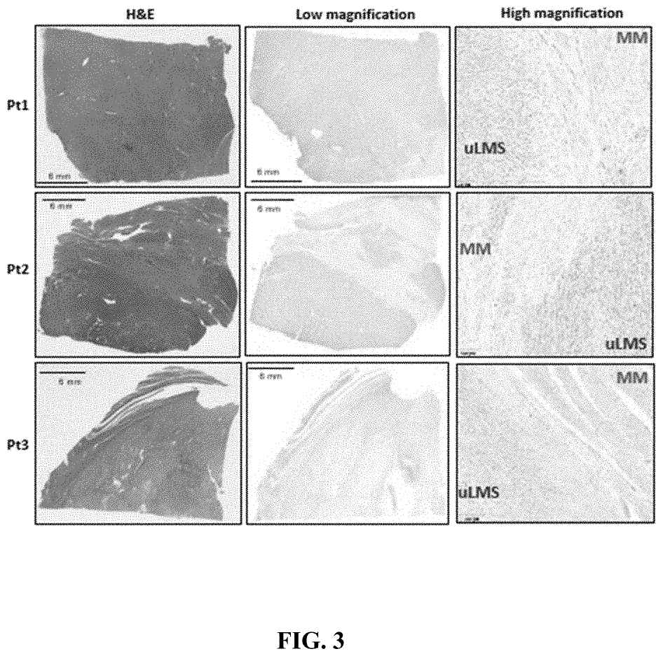

FIG. 1 demonstrates that the expression level of TTR appears to be upregulated in uLMS compared with adjacent myometrium, indicated by the increase in dark staining in the uLMS portions of the tissues, most readily seen at high magnification. Conversely, GPR179 appears to be downregulated in uLMS compared to adjacent myometrium tissues in patients 1 and 3 (FIG. 2). The comparative levels of expression of DUOX2 between uLMS and myometrium could not be conclusively determined by IHC (FIG. 3).

### Example 3

### Cell Lines

The immortalized human fibroid cell line (HuLM) and the uterine leiomyosarcoma (uLMS) cell line SK-UT1 and their culture conditions are described in Example 1.

The immortalized human uterine myometrial smooth muscle (UTSM) cell line are described in Example 1. The UTSM cell line is cultured as is described in Example 1 for the HuLM cell line.

### Enzyme-Linked Immunosorbent Assay (ELISA)

ELISA kits are measured following the manufacturing instructions of the following kits:

Human DUOX2 (Dual Oxidase-2) (My BioSource, San Diego, Calif., USA); ELISA kit catalog #MBS7251599 is a 96-well ELISA (sandwich ELISA) for the quantitative detection of human DUOX2 in samples. It detects levels of DUOX2 as low as 0.069 nanograms per ml.

Human Prealbumin ELISA kit (transthyretin) (ab108895) (Abcam, Waltham, Mass., USA). In the kit, PreAlbumin specific antibody are precoated onto 96-well plates and blocked. Standards or test samples are added to the wells and subsequently a pre-albumin specific biotinylated detection antibody is added and then followed by washing with wash buffer. Streptavidin-Peroxidase Conjugate is added, and unbound conjugates are washed away with wash buffer. TMB is then used to visualize the Streptavidin-Peroxidase enzymatic reaction.

GPR-179 Human Probable G-protein coupled receptor 179 (Biobool, Hong Kong, CN). Human GPR179 ELISA Kit Catalog No. E03813 allows for the in vitro quantitative determination of GPR179: concentrations in serum, plasma, tissue homogenates and cell culture supernates and other biological fluids.

Expression of the three candidate biomarkers in conditioning media (for growing human cell lines in vitro) is analyzed using ELISA.

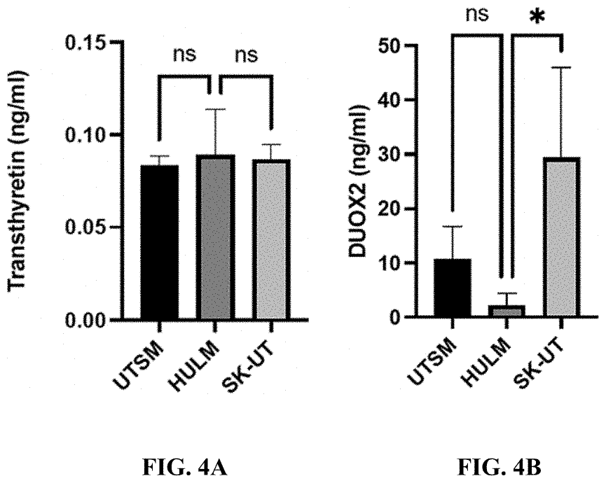

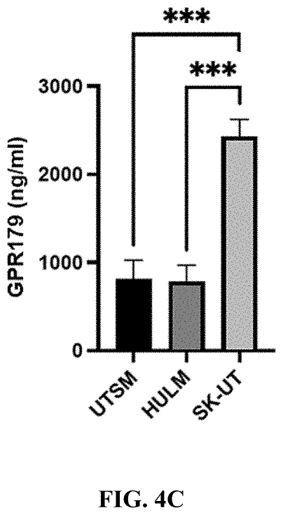

In FIG. 4, UTSM represents immortalized human uterine myometrial smooth muscle; HULM represents immortalized human fibroid cell line; and SK-UT represents a human leiomyosarcoma (uLMS) cell line. FIG. 4A shows a slight, but not significant, difference in transthyretin (TTR) levels in the growth media between the HULM and SK-UT tumor cell lines and the UTSM control. FIG. 4B shows a significant increase of DUOX2 level (two-fold) in the SK-UT leiomyosarcoma cell line medium relative to the HULM fibroid cell line medium. FIG. 4C shows a significant increase of GPR179 level in the SK-UT cell line growth medium relative to that of both UTSM and HULM (one-fold increase).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of differentiating a malignant tumor from a non-malignant tumor in a mammal having a tumor, the method comprising:
(a) providing a biological sample from the mammal;
(b) determining in the sample the level of one or more of: (1) transthyretin (TTR) RNA or protein, (2) dual oxidase 2 (DUOX2) RNA or protein, and (3) G-protein coupled receptor 179 (GPR179) RNA or protein;
(c) comparing the level of the one or more RNAs or proteins to the level of the one or more RNAs or proteins in a control biological sample; and
(d) determining the tumor is a malignant tumor if (i) the level of TTR RNA or protein in the sample is different than the level of TTR RNA or protein in the control, (ii) the level of DUOX2 RNA or protein in the sample is different than the level of DUOX2 RNA or protein in the control, or (iii) the level GPR179 RNA or protein in the sample is different than the level GPR179 RNA or protein in the control.

2. The method of claim 1, wherein the non-malignant tumor is a benign tumor.

3. The method of claim 1, wherein the biological sample is serum.

4. The method of claim 1, wherein the differentiation is between a malignant uterine tumor and a benign uterine tumor, wherein the malignant uterine tumor is a uterine leiomyosarcoma (LMS) and the benign uterine tumor is a uterine fibroid (UF).

5. The method of claim 4, wherein the tumor is determined to be an LMS tumor.

6. The method of claim 1, wherein (i) the level of TTR RNA or protein in the sample is higher than the level of TTR RNA or protein in the control, (ii) the level of DUOX2 RNA or protein in the sample is higher than the level of DUOX2 RNA or protein in the control, or (iii) the level GPR179 RNA or protein in the sample is different than the level of GPR179 RNA or protein in the control.

7. The method of claim 1, wherein (i) the level of TTR RNA or protein in the sample is higher than the level of TTR RNA or protein in the control, (ii) the level of DUOX2 RNA or protein in the sample is higher than the level of DUOX2 RNA or protein in the control, and (iii) the level GPR179 RNA or protein in the sample is different than the level of GPR179 RNA or protein in the control.

8. The method of claim 6, wherein the level is of RNA.

9. The method of claim 6, wherein the level is of protein.

10. The method of claim 1, wherein the differentiation is pre-operative.

11. The method of claim 1, wherein the level is of protein and is determined by quantitative mass spectrometry.

12. The method of claim 1, wherein the level is of protein and is determined by enzyme-linked immunosorbent assay.

13. The method of claim 1, wherein the mammal is a human female.

14. A method of treating a mammal having a malignant tumor, the method comprising:
(I) determining the tumor is a malignant tumor according to the method of claim 1; and
(II) treating the mammal with chemotherapy, radiotherapy, or surgery.

15. The method of claim 14, wherein the malignant tumor is a uterine leiomyosarcoma (LMS) tumor.

16. The method of claim 14, wherein (i) the level of TTR RNA or protein in the sample is higher than the level of TTR RNA or protein in the control, (ii) the level of DUOX2 RNA or protein in the sample is higher than the level of DUOX2 RNA or protein in the control, or (iii) the level GPR179 RNA or protein in the sample is different than the level of GPR179 RNA or protein in the control.

17. The method of claim 14, wherein (i) the level of TTR RNA or protein in the sample is higher than the level of TTR RNA or protein in the control, (ii) the level of DUOX2 RNA or protein in the sample is higher than the level of DUOX2 RNA or protein in the control, and (iii) the level GPR179 RNA or protein in the sample is different than the level of GPR179 RNA or protein in the control.

18. The method of claim 16, wherein the level is of RNA.

19. The method of claim 16, wherein the level is of protein.

20. The method of claim 16, wherein the mammal is a human female.
